(19) United States
(12) Patent Application Publication
(10) Pub. No.: US 2026/0263816 A1
(43) Pub. Date: Sep. 10, 2026

(54) MULTIPLE SECURE ENCRYPTION CHANNEL MEDICAL SYSTEM DESIGN

(71) Applicant: Medtronic, Inc., Minneapolis, MN (US)

(72) Inventors: Andrew T. Fried, Saint Paul, MN (US); Charles M. Nowell, Longwood, FL (US); Igor A. Simanovich, Plymouth, MN (US); Laura A. Skarie, Deephaven, MN (US); Saketh Karumuri, Maple Grove, MN (US); Kunal J. Paralikar, PhD, Roseville, MN (US)

(21) Appl. No.: 19/165,887

(22) PCT Filed: Mar. 28, 2024
(86) PCT No.: PCT/US2024/022015

§ 371 Date: Sep. 16, 2025

Related U.S. Application Data
(60) Provisional application No. 63/492,716, filed on Mar. 28, 2023.

(51) Int. Cl. A61N 1/372; H04L 9/32; H04L 67/12; H04W 76/15

(52) U.S. Cl. CPC A61N 1/37254; A61N 1/37247; H04L 9/32; H04L 67/12; H04W 76/15

(57) ABSTRACT

Systems, devices, and. techniques to communicate between external computing devices and medical devices. The systems may include two or more communication channels using one or more communication links between the medical device and the external computing devices. The two or more communication channels may transfer data, commands and other information over the same communication link using time multiplexing to share the link bandwidth between the communication channels. Each communication channel of the two or more channels may have a separate authentication level and be authorized to convey different levels of information. Each communication channel may be a secure communication channel and transfer information using information set associated with the authentication level based on encryption keys negotiated for the duration of a communication session.

This application is a PCT application that claims priority to, and the benefit of, U.S. Provisional Patent Application No. 63/492,716, filed Mar. 28, 2023, the entire contents of which is incorporated herein by reference.

### TECHNICAL FIELD

The disclosure relates to medical device communication, and more specifically to communication with a wearable or implantable medical device on a patient.

### BACKGROUND

Medical devices may be external or implanted and may be used to monitor patient signals such as cardiac activity, biological impedance and to deliver electrical stimulation therapy to patients via various tissue sites to treat a variety of symptoms or conditions such as chronic pain, tremor, Parkinson's disease, diabetes, epilepsy, urinary or fecal incontinence, sexual dysfunction, obesity, or gastroparesis and other conditions. In some examples, the medical devices may communicate with one or more external computing devices. The communications may include programming the device to operate according to patient condition as well as transmitting updates on the patient status to, for example, a clinician caring for the patient. In some examples, medical devices may include a rechargeable electrical power source, or may be powered directly by transmitting energy through tissue. In other examples, a medical device may receive power from a primary cell battery (non-rechargeable) or from line power.

### SUMMARY

In general, the disclosure describes systems, devices, and techniques to communicate between external computing devices and devices such as a wearable or implantable medical device. The communication for the systems of this disclosure may include two or more communication channels using one or more communication links between the medical device and one or more external computing devices. In some examples, two or more communication channels may transfer data, commands and other information over a communication link using time division multiplexing to share the link bandwidth between the different communication channels. In some examples, each communication channel of the two or more channels may have a separate authentication level and be authorized to convey different levels of information. For example, the authentication level for a power transfer unit, e.g., a recharger, may only authorize sending and receiving queries and status information about the power transfer function, such as the electrical energy storage level of a battery, or a power transfer rate. The authentication level for a programming device, using the same link but a different channel, may instead communicate commands and information that change the operation of the medical device worn by, or implanted in, the patient, such as a parameters that define a therapy delivery program. In some examples the communication channels may be each be secure communication channels with separate encryption handshaking. In other examples, the medical device may have multiple radios each of which could have multiple communication/encryption channels, and may communicate using the same, or different, communication protocols.

In one example, this disclosure describes an implantable medical device comprising communication circuitry configured to: establish a first communication channel with a first external computing device on a communication link; establish a second communication channel with a second external computing device on the communication link, wherein the first communication channel transfers first information at a first authentication level on the communication link, and wherein the second communication channel transfers second information at a second authentication level different than the first authentication level on the same communication link.

In another example, this disclosure describes a method comprising establishing, by communication circuitry of an implantable medical device, a first communication channel with a first external computing device on a communication link; establishing, by the communication circuitry, a second communication channel with a second external computing device on the communication link, wherein the first communication channel transfers first information at a first authentication level, and wherein the second external computing device transfers second information on the second communication channel via the first external computing device at a second authentication level different than the first authentication level.

In another example, this disclosure describes a non-transitory computer-readable storage medium comprising instructions that, when executed, cause one or more processors of a computing device to: cause communication circuitry of an implantable medical device to establish a first communication channel with a first external computing device on a communication link; cause communication circuitry of the implantable medical device to establish a second communication channel with a second external computing device on the communication link, wherein the first communication channel transfers first information at a first authentication level, and wherein the second external computing device transfers second information on the second communication channel via the first external computing device at a second authentication level different than the first authentication level.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating a medical system of this disclosure that includes an implantable medical device located near an ankle of a patient.

FIG. 2 is a block diagram illustrating an example communication configuration for a system according to one or more techniques of this disclosure.

FIG. 3 is a conceptual diagram illustrating a medical system of this disclosure that includes an implantable medical device located near a pelvis of a patient.

FIG. 4 is a block diagram illustrating example components of the implantable medical device of FIGS. 1-3.

FIG. 5 is a block diagram of an example an external computing device of FIGS. 1 and 3.

FIG. 6 is a flow chart illustrating an example mode of operation of processing circuitry controlling the communication circuitry of the systems of this disclosure.

### DETAILED DESCRIPTION

The disclosure describes systems, devices, and techniques to communicate between external computing devices and medical devices such as a wearable or implantable medical device. In some examples, only one external instrument may have been configured to communicate with a medical device for a patient, (e.g., one clinician programmer, one patient programmer, one recharger, or similar external device). In some examples the one external instrument may have been a recharger used as a communicator. In some examples, a patient may have the rechargers strapped near the medical device, e.g., to the ankle of the patient for a tibial implanted device, when the system is using a Clinician Programmer or Patient Programmer to communication to the medial device. Allowing only one device to set up a communication link with a single communication channel may have been one technique to ensure communication security.

In contrast, the system of this disclosure may allow for multiple secure communication channels to be established and maintained. For example, the communication for the systems of this disclosure may include two or more communication channels using one or more communication links between the medical device and the external computing devices. In some examples, two or more communication channels may transfer data, commands and other information over the same communication link using time division multiplexing to share the link. In some examples, each communication channel of the two or more channels may have a separate authentication level and be authorized to convey different levels of information. In this way, different authentication levels may be associated with different actions or features that the device can perform with a request made via communication with that authentication level. In some examples, each communication channel may be a secure communication channel and transfer information using an information set associated with the authentication level based on encryption keys negotiated for the duration of a communication session.

As one example, there are use cases in which the recharger may be operating using closed loop recharging, which may include periodic communication to the medical device for the patient. At the same time, the clinician and/or patient may want to communicate with the medical device using an external programmer. The secure communication channel feature of the systems of this disclosure may allow for all communication to the medical device to occurs over a single communication link to the medical device, and only one telemetry command may be sent at a time. However, the encryption used for the recharger commands may be different from the encryption used for the programmer commands. Also, encryption may be unique to each communication channel for each communication session. The secure communication channels of the systems of this disclosure provide advantages over other systems in that the systems of this disclosure may allow for a secure communication sessions to be setup and maintained for one, two or more external devices. The multiple communication channels over the communication link during the communication session may enable the medical device to receive and respond to telemetry commands from more than one external device without the overhead of setting up secure communication every time the system needs to toggle between communication for each external device, e.g., to toggle between a recharger and communication from a programmer, as was used in other examples described above. In other words, the multiple communication channels appear to be simultaneous, from the perspective of the user. Other previously developed systems have only allowed one activity by one external computing device at a time, whereas this approach allows multiple user-sessions to be concurrently maintained and acted upon. Thus, the multiple secure channels of this disclosure may improve communication latency and user experience.

In this manner, the systems of this disclosure may enable new use scenarios that have not been possible in the past such as clinician programming and patient programming simultaneously for patient training, patient remote usage using a dedicated patient programmer simultaneous with usage of a connected tablet computer or mobile phone application sending data on another secure channel with the medical device. Other advantages of the system of this disclosure may include recharging simultaneous with patient remote usage, e.g., the patient remote may be connected on another separate secure channel with the medical device while a recharger is actively providing closed loop recharging. Similarly recharging while securely communicating with the medical device using an external computing device running an application, e.g., a tablet, laptop, or mobile phone.

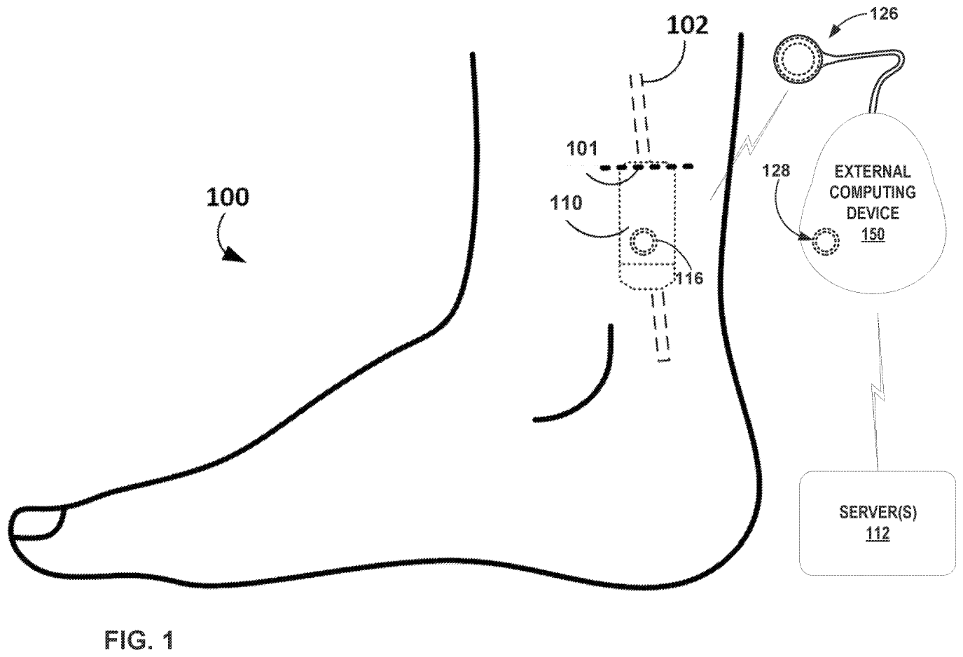

The example of FIG. 1 is a conceptual diagram illustrating an example medical system 100 of this disclosure that includes an implantable medical device located near an ankle of a patient. The example of system 100 in FIG. 1 includes an implantable medical device (IMD) 110, external computing device 150, and one or more servers 112.

In some examples, external computing device 150 may also be referred to as programmer 150, external recharging device 150 or recharger 150. External computing device 150 as shown in FIG. 1 includes one or more antenna, such as antenna 126 and antenna 128. External computing device 150 may be used to program or adjust settings of IMD 110 and may also recharge an electrical energy storage device, such as a battery, of IMD 110 (not shown in FIG. 1). External computing device 150 may also communicate with one or more servers 112. In other examples, external computing device 150 may also include a mobile phone, tablet computer, a wearable computing device or similar computing device that includes processing circuitry configured to execute programming instructions to communicate with IMD 110 and/or servers 112. Such a computing device may communicate with IMD 110 to adjust therapy and/or sensing parameters, download recorded data, and the other functions described in this disclosure.

IMD 110 may include sensing circuitry configured to detect biological signals from the patient; electrical stimulation circuitry configured to deliver electrical stimulation to target tissue of the patient, communication circuitry, processing circuitry configured to control the operation of circuitry of IMD 110 and other circuitry (not shown in FIG. 1) configured to perform the functions described in this disclosure. IMD 110 may output the sensed data via communication circuitry (not shown in FIG. 1).

The communication circuitry may be configured to communicate with external computing devices in system 100. In some examples, the communication circuitry may establish a first communication channel with a first external computing device on a communication link, such as with external computing device 150. In some examples, the communication circuitry may also establish a second communication channel with a second external computing device on the same communication link. The communication link may be a communication protocol that can only send data to one device at any given time. The second external communication device may include servers 112, a mobile phone, a patient programmer or similar computing device. The communication circuitry of IMD 110, as well as communication circuitry of external computing device 150, servers 112 and other devices of system 100 may be configured to establish the first communication channel and the second communication channel on the communication link using time division multiplexing. By multiplexing the information for each communication channel over the same communication link, the respective data for each communication channel may be interleaved together over time.

The first communication channel may transfer information at a first authentication level, while the second communication channel may transfer information at a second authentication level. In some examples, the first communication channel is a secure communication channel established based on a first encryption key, and the second communication channel is a secure communication channel established based on a second encryption key. IMD 110 may communicate via a wireless protocol (e.g., Bluetooth™, Bluetooth Low Energy (BLE), or another protocol such as inductive communication or a protocol using the Medical Implant Communication System (MICS) band) to a number of different instruments, such as, for example, an additional medical device, a patient programmer, a clinician programmer, a programming fob, or another device.

In some examples, one authentication level may provide access to more capabilities of IMD 110 than the other authentication levels. In the example in which external computing device 150 is a recharger, external computing device 150 may establish the first communication channel in which the authentication level allows only communication related to power transfer, as described above. Communication related to power transfer may limit recharger 150 to receive and transfer information such as battery current, battery discharge level, power transfer efficiency and similar system metrics and information. A variety of system metrics may be available to external computing device 150 from computations of power and heat and from metrics communicated from IMD 110. Processing circuitry of system 100, e.g., processing circuitry of external computing device 150, processing circuitry of servers 112, and/or processing circuitry of IMD 110, may calculate any of the values described herein. These metrics may include but are not limited to: battery current, power transfer efficiency, IMD efficiency and other similar metrics. Analysis of system characterization data that the IMD efficiency, which may be measured by IMD 110 and communicated to external computing device 150, may be an example indicator of when the recharger primary coil 126 is concentric with secondary coil 116.

As noted above, at the same time, IMD 110 may communicate with one or more other devices over one or more additional communication channels at a different authentication level. Each communication channel may have a separate authentication level and may have separate encryption keys. For example, a clinical programmer may establish a communication channel with an authentication level that allows the clinical programmer to change operating parameters, set or change therapy modes, set up patient data collection, receive detailed patient sensing data, and other similar functions. In some examples the clinician programmer may be near IMD 110, e.g., in the same room. In other examples, the clinician programmer may be at a remote location, operated by a caregiver, and communicate via servers 112, or some other communication device. Similarly, a patient programmer may communicate at a third authentication level with a more limited set of functions, such as may be able to cause IMD 110 to increase or decrease stimulation amplitude and intensity, but be unable to change a programmed therapy protocol. In other words a first authentical level may be configured to communicate with a first information set, e.g., a patient programmer, or external device 150 operating as a recharger. The second authentication level on the second communication channel, e.g., a clinician programmer, may be configured to communicate with an expanded information set with more or different elements than the first information set. In some examples, there will be times when the external device 150 may not even have a communication channel of its own and may act only as a relay of communications from one or more server(s) 112. Some examples of items in an information set may include: data, operating status, operating commands, and therapy parameters that define delivered therapy such as amplitude, pulse width, frequency, burst length, and other parameters that define therapy. Some examples of items in an information set may also include configuration of sense circuitry in the medical device, identifying information e.g., related to identifying the patient, the computing device, location and similar identification information, power information, firmware update commands, memory access commands, and configuration of closed loop therapy algorithms.

Some other examples of roles and associated authentication levels for communication channels may include any one or more of clinician, patient, recharger, firmware update, security level change, and remote. In some examples, the programming instructions for processing circuitry of system 100 may prevent conflicting commands and other information transfer between the two or more communication channels, which may have different roles. For example, processing circuitry of system 100, e.g., of external computing device 150, IMD 110 or some other processing circuitry, may prevent a patient programmer from trying to decrease a parameter at the same time a clinician programmer is trying to increase the parameter. In other examples, one or more roles may be restricted based on the role of an established communication channel, e.g., the patient programmer may not establish a communication channel during a firmware update. In other examples, the number of channels may be restricted, e.g., only two channels may connect at the same time, or no more than three channels may connect at the same time.

In some examples, computing devices of system 100 may be configured to identify, open, and close a secure communications channel to and from IMD 110. Each secure channel may be independent from each other in channel establishment, generated encryption keys, and authorization roles, as noted above. In some examples, each channel may have a channel validity timer, which is set to a configured value at the time the external computing device, e.g., external computing device 150, establishes a channel with IMD 110. In other words, the communication circuitry may be configured to establish the secure communication based on an encryption key for the duration of a communication session. The encryption for the communication channel, and the associated authorization level, may be time limited to the communication session for each channel.

In some examples, the channel validity timer may be reset upon each successful decryption of subsequent messages, or based on some other communication event, effectively extending the communication session. When the timer for a channel expires, that channel may be invalidated and subsequent commands may not be processed. In the event a channel timer expires, a user for the external computing device may request to open a new channel to issue subsequent commands or the external computing device may reopen a new channel automatically and in the background.

In some examples, authorization to transfer information may be granted upon a successful channel open command. The authorization is valid for the duration of the channel validity timer. Channel validity may be set to a configured value of ten minutes, five minutes, twenty minutes, or any number of other values. In some examples, the channel validity time for each channel may be the same, or may be different, from other channels and based on authentication level. In some examples, upon successfully decrypting a user command processing circuitry of one or more of the computing devices of system 100 may reset the channel validity timer. The channel validity timer may count down from the set time and at zero, may cause the processing circuitry to revoke the channel validity and authorization. After the channel validity and authorization have been revoked, any subsequent commands may result in a non-decryptable message and commands are not executed. The programming instructions for the processing circuitry of system 100 may not respond to application commands and other messages when channel encryption keys are invalid/expired. In some examples, the processing circuitry may set a register and/or output an error message. In some examples, when IMD 110 is reset or security level is changed the communication channels may also be reset, which may clear the channel encryption keys and set the channel state to “unused.” Each computing device may then re-negotiate encryption keys to establish and continue secure communication over the established communication channel.

In the example of a rechargeable power source, the rechargeable power source of IMD 110 may include one or more capacitors, batteries, or other components, e.g., chemical, or electrical energy storage devices (not shown in FIG. 1). Example batteries may include lithium-based batteries, nickel metal-hydride batteries, or other materials. The rechargeable power source may be replenished, refilled, or otherwise capable of increasing the amount of energy stored after energy has been depleted. The energy received from secondary coil 116 may be conditioned and/or transformed by a charging circuit. The charging circuit may then send an electrical signal used to charge the rechargeable power source when the power source is fully depleted or only partially depleted.

External computing device 150 may be used to recharge the rechargeable power source within IMD 110 implanted in the patient. External computing device 150 may be a hand-held device, a portable device, or a stationary charging system. External computing device 150 may include components necessary to charge IMD 110 through tissue of the patient. External computing device 150 may include an internal energy transfer coil 128 and external energy transfer coil 126, also referred to as primary coil 126 or primary coil 128. In other examples, external computing device may only include internal primary coil 128 and omit the use of external primary coil 126, may have multiple internal and external coils, or may omit internal primary coil 128 and use external primary coil 126.

External computing device 150 may include a housing to enclose operational components such as a processor, memory, user interface, telemetry circuitry, power source, and charging circuit configured to transmit energy to secondary coil 116 via energy transfer coil 126 and/or 128. Although a user may control the recharging process with a user interface of external computing device 150, external computing device 150 may alternatively be controlled by another device, e.g., an external programmer, a computing device of servers 112, where such servers may include a tablet computer, laptop, or other similar computing device. In other examples, external computing device 150 may be integrated with an external programmer, such as the patient programmer carried by the patient.

External computing device 150 and IMD 110 may utilize any wireless power transfer techniques that are capable of recharging the power source of IMD 110 when IMD 110 is implanted within the patient. In some examples, system 100 may utilize inductive coupling between primary coils (e.g., energy transfer coil 128) and secondary coils (e.g., secondary coil 116) of external computing device 150 and IMD 110. In inductive coupling, energy transfer coil 128 is placed near implanted IMD 110 such that energy transfer coil 128 is aligned with secondary coil 116 of IMD 110. External computing device 150 may then generate an electrical current in energy transfer coil 128 based on a selected power level for charging the rechargeable power source of IMD 110.

When the primary and secondary coils are aligned, or partially aligned, the electrical current in the primary coil may magnetically induce an electrical current in secondary coil 116 within IMD 110. Since the secondary coil is associated with and electrically coupled to the rechargeable power source, the induced electrical current may be used to increase the voltage, or charge level, of the rechargeable power source. Although inductive coupling is generally described herein, any type of wireless energy transfer may be used to transfer energy between external computing device 150 and IMD 110.

Energy transfer coil 126 and 128 may include a wound wire (e.g., a coil) (not shown in FIG. 1). The coil may be constructed of a wire wound in an in-plane spiral (e.g., a disk-shaped coil). In some examples, this single or even multi-layers spiral of wire may be considered a flexible coil capable of deforming to conform with a non-planar skin surface. The coil may include wires that electrically couple the flexible coil to a power source and a charging module configured to generate an electrical current within the coil. Energy transfer coil 128 may be external of the housing of external computing device 150 such that energy transfer coil 128 can be placed on the skin of the patient proximal to IMD 110. In some examples, energy transfer coil 128 may be disposed on the outside of the housing or even within housing.

Either primary coil 126 and/or 128 of system 100 may include a heat sink device (not shown in FIG. 1). In the example of system 100, external computing device 150 is the power transmitting unit and IMD 110 is the power receiving unit. IMD 110 may be in a flipped or non-flipped position.

As noted above external computing device 150 may also be referred to as recharger 150. External computing device 150 may include a user interface to receive control inputs from a user, such as the patient, medical professional, or other caregiver. External computing device 150, and any computing device of system 100, may include a touch-screen user interface. The user interface of external computing device 150 may also provide information to a user, including whether IMD 110 is ON and delivering therapy, whether external computing device 150 is wirelessly communicating with IMD 110 and similar information.

The example of FIG. 1 is a side view of a patient's leg showing IMD 110 as a leadless neurostimulation device near the ankle adjacent to the tibial nerve 102. IMD 110 can be implanted through the patient's skin and cutaneous fat layer via a small incision 101 (e.g., about one to three cm) above the tibial nerve on a medial aspect of the patient's ankle. While incision 101 is shown approximately horizontal to the length of the tibial nerve, other incisions or implantation techniques could be used according to physician preference. The example of FIG. 1 describes a neurostimulation implantable medical device for tibial nerve stimulation. In other examples, the techniques of this disclosure may apply to other rechargeable devices, such as implantable neurostimulation system for use in spinal cord stimulation therapy, deep brain stimulation, as well as to other types of medical devices without limitation. In this disclosure, IMD 110 may referred to as an implantable medical device (IMD) 110 or, in the example of a neurostimulation medical device, may be referred to as implantable neuro stimulator (INS) 110.

IMD 110 may be positioned adjacent to the region defined by flexor digitorum longus and soleus in which tibial nerve 102 is contained and implanted adjacent and proximal to a fascia layer. One or more electrodes of IMD 110 may face toward tibial nerve 102. Though not shown in FIG. 1, IMD 110 may also connect to one or more leads comprising one or more electrodes (not shown in FIG. 1).

IMD 110 may be constructed of any polymer, metal, or composite material sufficient to house the components of IMD 110. In this example, IMD 110 may be constructed with a biocompatible housing, such as titanium or stainless steel, or a polymeric material such as silicone or polyurethane, and surgically implanted at a site in patient near the tibial nerve, in some examples, while in other examples, implanted near the pelvis, abdomen, or buttocks. The housing of IMD 110 may be configured to provide a hermetic seal for components, such as a rechargeable power source. In addition, the housing of IMD 110 may be selected of a material that facilitates receiving energy to charge the rechargeable power source.

During normal operation after implantation, an electrical stimulation signal may be transmitted between one or more electrodes through the fascia layer. The electrical signal may be used to stimulate tibial nerve 102 which may be useful in the treatment of overactive bladder (OAB) symptoms of urinary urgency, urinary frequency and/or urge incontinence, or fecal incontinence.

One type of therapy for treating bladder dysfunction includes delivery of electrical stimulation to a target tissue site within a patient to cause a therapeutic effect during delivery of the electrical stimulation. For example, delivery of electrical stimulation from IMD 110 to a target therapy site, e.g., a tissue site that delivers stimulation to modulate activity of a tibial nerve, spinal nerve (e.g., a sacral nerve), a pudendal nerve, dorsal genital nerve, an inferior rectal nerve, a perineal nerve, or branches of any of the aforementioned nerves, may provide a therapeutic effect for bladder dysfunction, such as a desired reduction in frequency of bladder contractions. In some cases, electrical stimulation of the tibial nerve may modulate afferent nerve activities to restore urinary function.

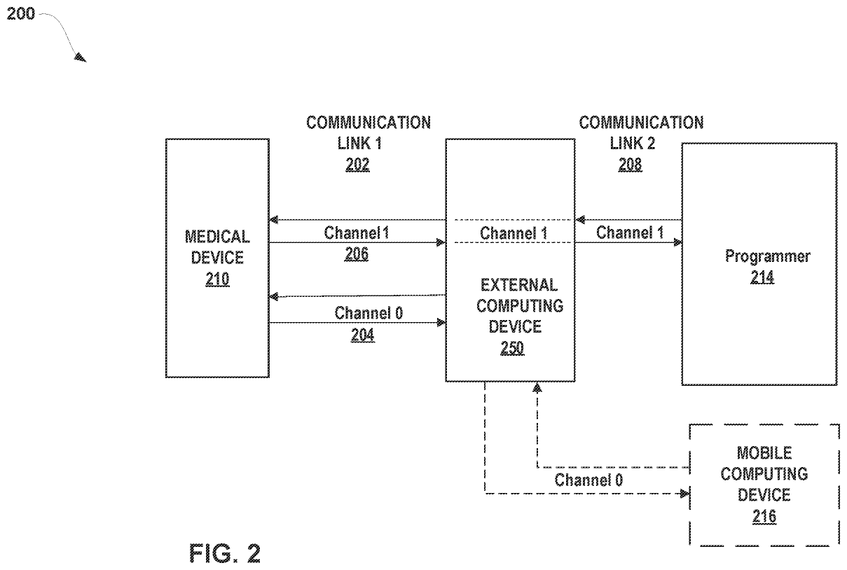

FIG. 2 is a block diagram illustrating an example communication configuration for a system according to one or more techniques of this disclosure. System 200 of FIG. 2 is an example of system 100 described above in relation to FIG. 1 and may have the same characteristics and functions as described above for system 100. In some examples medical device 210, also referred to as device 210, may be an implantable or wearable device as described above in relation to FIG. 1. In other examples, medical device 210 may also include medical systems that are capital equipment that may be neither body worn nor implantable. Some examples of capital equipment may include a surgical navigation system, a blood oxygen monitoring system, a robotic surgery system and other types of capital equipment. For example, a robotic surgery system may establish a communication channel with a first provider, e.g., a surgeon in an international location, as well as with a provider that is local and may be in the same operating room as the robotic surgery system.

Communication circuitry of device 210 may be configured to establish communication channel 0 (204) with external computing device 250 on communication link 1 (202). The communication circuitry of device 210 may also establish a second communication channel 1 (206) with a second external computing device on the same communication link 1 (202), e.g., with programmer 214. Device 210 may further establish additional communication channels on communication link 1 (202) with other computing devices, e.g., servers 112 or other computing devices as described above in relation to FIG. 1. Communication link 1 (202) may thus be a single communication protocol that defines data flow between Device 210 and external computing device 250. Therefore, each communication channel can use a respective portion of the data transmitted over communication link 1 (202). Although the example of FIG. 2 only illustrates two channels and two communication links to simplify the description, communication link 1 (202) may include any number of channels, e.g., three or more channels.

In some examples, processing circuitry of computing device 250 may manage opening and establishing the communication channels between device 210 and other computing devices. In some examples, external computing device 250 may establish first communication channel 0 (204) directly to device 210. External computing device 250 may further establish communication channel 1 (206) directly to the device 210. In the example of system 200 in FIG. 2, programmer 214 may transfer information to and from device 210 on communication channel 1 (206) via external computing device 250. In some examples the communication over channel 1 (206) may be described as end-to-end encryption. For end-to-end encryption, external computing device 250 may not be configured to decrypt the information that programmer 214 is sending and receiving over channel 1 (206) because external computing device 250 may not have the encryption key for authentication level of channel 1 (206).

In some examples, such as shown in FIG. 2, communication link 1 (202) between external computing device 250 and device 210 is a first communication link and programmer 214 may communicate with external computing device 250 via a second communication link, e.g., communication link 2 (208). In some examples, communication link 1 (202) may operate with the same protocol as communication link 2 (208). In other examples, communication link 1 (202) may operate with a different protocol from communication link 2 (208), e.g., communication link 1 (202) may use a first protocol, while communication link 2 (208) may use BLE or some other wired or wireless communication protocol.

In other examples, external computing device 250 may establish communication link 1 (202), but may establish a communication channel between medical device 210 for a second external computing device, rather than a communication channel between external computing device 250 and medical device 214. In other words, instead of establishing its own communication channel, external computing device 250 may provide the interface for two or more secondary computing devices, e.g., programmer 214 and mobile computing device 216. In other examples, (not shown in FIG. 2) the secondary computing device may include a patient programmer and clinical programmer, a patient programmer and remote programming, or some other combination of communication channels set up “simultaneously” in the same session.

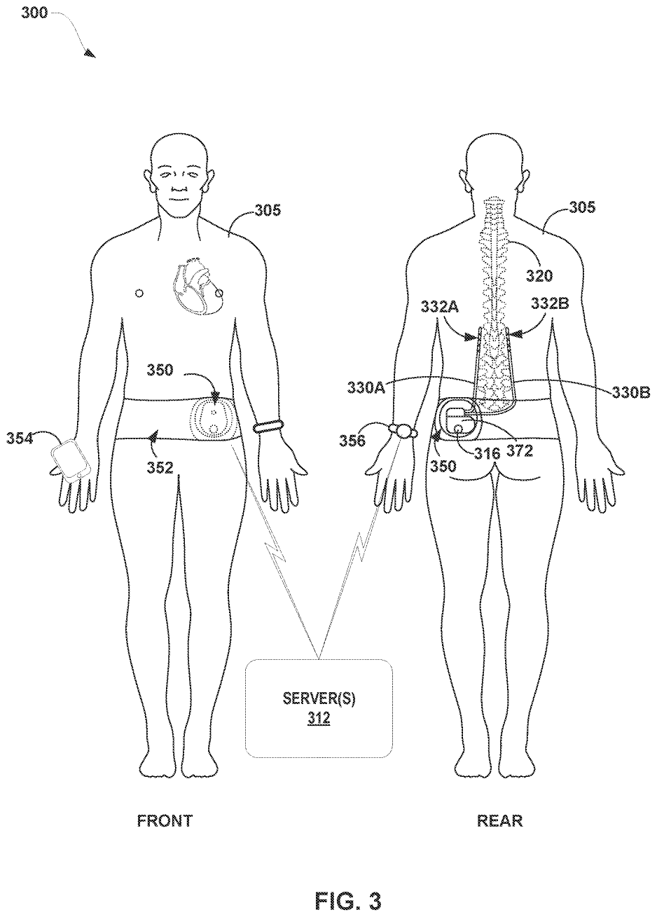

FIG. 3 is a conceptual diagram illustrating an example system 300 that includes an IMD 372 configured to deliver spinal cord stimulation (SCS) therapy and an external computing device 350, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices. In the example of FIG. 3, system 300 includes IMD 372 with antenna 316, external computing device 350 and servers 312, which are, respectively examples of IMD 110 with antenna 16, external computing device 150 and servers 112 described above in relation to FIG. 1 and may have the same or similar functions and characteristics.

As shown in FIG. 3, system 300 includes an IMD 372, leads 330A and 330B, and external computing device 350 shown in conjunction with a patient 305, who is ordinarily a human patient. In the example of FIG. 3, IMD 372 is an implantable electrical stimulator that is configured to generate and deliver electrical stimulation therapy to patient 305 via one or more electrodes of electrodes 332A and 332B, respectively on leads 330A and/or 330B (collectively, “leads 330”), e.g., for relief of chronic pain or other symptoms. In other examples, IMD 372 may be coupled to a single lead carrying multiple electrodes or more than two leads each carrying multiple electrodes, or leadless, as in the example of IMD 110 depicted in FIG. 1. IMD 372 may include an electrical connector configured to connect to the electrical leads, e.g., in the header of IMD 372.

IMD 372 may be a chronic electrical stimulator that remains implanted within patient 305 for weeks, months, or even years. In other examples, IMD 372 may be a temporary, or trial, stimulator used to screen or evaluate the efficacy of electrical stimulation for chronic therapy. In one example, IMD 372 is implanted within patient 305, while in another example, IMD 372 is an external device coupled to percutaneously implanted leads.

In the example of FIG. 3, external computing device 350 may be placed near IMD 372 to communicate and/or transfer power to IMD 372. In some examples, external computing device 350 may be held in place by a belt or straps 352. In some examples belt 352 may include a pouch that accepts external computing device 350. Any of the computing devices of system 300 may include a user interface. Examples of the user interface may include indicator lights, audio feedback, or graphics displayed on a graphical user interface (GUI) such as a tablet computer, smart phone 354, wearable computing device 356 or similar device.

As described above in relation to FIGS. 1 and 2, in some examples, a user, such as a clinician or patient 305, may interact with a user interface of an external computing device, such as external computing device 350 or server 312, to program IMD 372, download collected patient data, and similar interactions. Communication circuitry of system 300, located on any of IMD 372 and the external computing devices of system 300 may establish one or more communication channels on a communication link. The communication channels may share the communication link using time division of the link bandwidth. Each communication channel may have a separate authentication level and be configured to transfer information based on an associated information set.

Programming of IMD 372 may refer generally to the generation and transfer of commands, programs, or other information to control the operation of IMD 372. In this manner, IMD 372 may receive the transferred commands and programs from external computing device 350 to control stimulation, such as electrical stimulation therapy (e.g., informed pulses), control stimulation (e.g., control pulses), haptic stimulation, sensing and other operating parameters. When operating as a recharging device, external computing device 350 may communicate to IMD 372 with a more limited information set than a patient programmer or clinician programmer.

For example, a clinician programmer may transmit therapy stimulation programs, evoked compound action potential (ECAP) test stimulation programs, stimulation parameter adjustments, therapy stimulation program selections, ECAP test program selections, user input, or other information to control the operation of IMD 372 as described above in relation to FIGS. 1 and 2. A patient programmer device, or wearable computing device 356 and mobile computing device 354 with processing circuitry executing an application configured to control the operation of IMD 372, may communication with a more limited information set and at a different authentication level than a clinical programmer.

As described above in relation to FIG. 1, information may be transmitted between external computing devices of system 300 and IMD 372. Therefore, IMD 372 and the external computing devices may communicate via wireless communication using any techniques known in the art. Examples of communication techniques may include, for example, radiofrequency (RF) telemetry and inductive coupling, but other techniques are also contemplated. In some examples, external computing device 350 includes a communication head, e.g., antenna 26 depicted in FIG. 1, that may be placed proximate to the patient's body near the IMD 372 implant site to improve the quality or security of communication between IMD 372 and external computing device 350. Communication between the external computing devices of system 300 and IMD 372 may occur during power transmission or separate from power transmission.

In some examples, the stimulation signals, or pulses, may be configured to elicit detectable ECAP signals that IMD 372 may use to determine the posture state occupied by patient 305 and/or determine how to adjust one or more parameters that define stimulation therapy. The processing circuitry of IMD 372 may cause the stimulation signals to also deliver haptic stimulation, e.g., stimulation above a perception threshold of patient 305, to provide patient feedback, such as feedback on the quality of alignment between the primary and secondary coils.

This disclosure will focus on a device used for spinal cord stimulation, as shown in the example of FIG. 3 to simplify the description. However, the techniques of this disclosure may also apply to other devices, including wearable devices that may be located elsewhere on patient 305. Some examples may include devices located near the head for deep brain stimulation (DBS), near the tibial region as in the example of FIG. 1, near the heart for cardiac therapy and/or monitoring, and other locations.

In other words, although in one example IMD 372 takes the form of an SCS device, in other examples, IMD 372 takes the form of any combination of DBS devices, implantable cardioverter defibrillators (ICDs), pacemakers, cardiac resynchronization therapy devices (CRT-Ds), left ventricular assist devices (LVADs), implantable sensors, orthopedic devices, or drug pumps, as examples. Moreover, techniques of this disclosure may be used to determine parameters that affect stimulation thresholds (e.g., perception thresholds and detection thresholds) associated any one of the aforementioned IMDs and then use a stimulation threshold to inform the intensity (e.g., stimulation levels) of therapy. For example, changing stimulation parameters such as the number of pulses in a burst, the number of bursts over a duration, the pulse width of a pulse in a burst, the ON-time, the OFF-time, a pattern of pulses over a duration and other parameters may change the intensity as well as the efficacy of the therapy to relieve the symptoms.

As with IMD 110 described above in relation to FIG. 1, IMD 372 may be constructed of any polymer, metal, or composite material sufficient to house the components of IMD 372 (e.g., components illustrated in FIG. 2) within patient 305. In this example, IMD 372 may be constructed with a biocompatible housing, such as titanium or stainless steel, or a polymeric material such as silicone, polyurethane, or a liquid crystal polymer, and surgically implanted at a site in patient 305 near the pelvis, abdomen, or buttocks. In other examples, IMD 372 may be implanted within other suitable sites within patient 305, which may depend, for example, on the target site within patient 305 for the delivery of electrical stimulation therapy. The outer housing of IMD 372 may be configured to provide a hermetic seal for components, such as a rechargeable or non-rechargeable power source. In addition, in some examples, the outer housing of IMD 372 is selected from a material that facilitates receiving energy to charge the rechargeable power source.

IMD 372 may deliver electrical stimulation energy, which may be constant current or constant voltage pulses, for example, to one or more target tissue sites of patient 305 via one or more electrodes 332A and 332B (collectively electrodes 332) of implantable leads 330. In the example of FIG. 3, leads 330 carry electrodes that are placed adjacent to the target tissue of spinal cord 320. One or more of electrodes 332 may be disposed at a distal tip of a lead 330 and/or at other positions at intermediate points along the lead. Leads 330 may be implanted and coupled to IMD 372. Electrodes 332 may transfer electrical stimulation generated by an electrical stimulation generator in IMD 372 to tissue of patient 305. Electrodes 332 may also sense bioelectrical signals of patient 305.

Although leads 330 may each be a single lead, lead 330 may include a lead extension or other segments that may aid in implantation or positioning of lead 330. In some other examples, IMD 372 may be a leadless stimulator with one or more arrays of electrodes arranged on a housing of the stimulator rather than leads that extend from the housing, as shown in IMD 110 of FIG. 1. In addition, in some other examples, system 300 may include one lead or more than two leads, each coupled to IMD 372 and directed to similar or different target tissue sites.

Electrodes 332A and 332B of leads 330 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 330 will be described for purposes of illustration.

The deployment of electrodes 332A and 332B via leads 330 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 330 are linear leads having 8 ring electrodes along the axial length of the lead. In another example, the electrodes are segmented rings arranged in a linear fashion along the axial length of the lead and at the periphery of the lead.

The stimulation parameter set of a therapy stimulation program that defines the stimulation pulses of electrical stimulation therapy by IMD 372 through the electrodes of leads 330 may include information identifying which electrodes have been selected for delivery of stimulation according to a stimulation program, the polarities of the selected electrodes, i.e., the electrode combination for the program, voltage or current amplitude, pulse frequency, pulse width, pulse shape of stimulation delivered by the electrodes. These stimulation parameters values that make up the stimulation parameter set that defines pulses may be predetermined parameter values defined by a user and/or automatically determined by system 300 based on one or more factors or user input.

Similarly, sensing bioelectrical signals may use a variety of combinations of electrodes on leads 330, the housing of IMD 372, or other sensors connected directly or indirectly to IMD 372. In some examples IMD 372 may measure and detect other bioelectrical signals from patient 305 including cardiac activity, thoracic impedance, water retention and other signals. In some examples, lead 330 includes one or more sensors configured to allow IMD 372 to monitor one or more parameters of patient 305, such as patient activity, pressure such as blood pressure, temperature, or other characteristics. The one or more sensors may be provided in addition to, or in place of, therapy delivery by lead 330.

Although FIG. 3 is directed to SCS therapy, e.g., used to treat pain, in other examples system 300 may be configured to treat any other condition that may benefit from electrical stimulation therapy. For example, system 300 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 300 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 305. In other examples, IMD 372 takes the form of any combination of deep brain stimulation (DBS) devices, implantable cardioverter defibrillators (ICDs), pacemakers, cardiac resynchronization therapy devices (CRT-Ds), left ventricular assist devices (LVADs), implantable sensors, orthopedic devices, drug pumps and so on.

IMD 372 is configured to deliver electrical stimulation therapy to patient 305 via selected combinations of electrodes carried by one or both of leads 330, alone or in combination with an electrode carried by or defined by an outer housing of IMD 372. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle. In the example illustrated by FIG. 3, the target tissue is tissue proximate spinal cord 320, such as within an intrathecal space or epidural space of spinal cord 320, or, in some examples, adjacent nerves that branch off spinal cord 320. Leads 330 may be introduced into spinal cord 320 in via any suitable region, such as the thoracic, cervical, or lumbar regions. Stimulation of spinal cord 320 may, for example, prevent pain signals from traveling through spinal cord 320 and to the brain of patient 305. Patient 305 may perceive the interruption of pain signals as a reduction in pain and, therefore, efficacious therapy results. In other examples, stimulation of spinal cord 320 may produce paresthesia which may be reduce the perception of pain by patient 305, and thus, provide efficacious therapy results.

IMD 372 is configured to generate and deliver electrical stimulation therapy to a target stimulation site within patient 305 via the electrodes of leads 330 to patient 305 according to one or more therapy stimulation programs. A therapy stimulation program defines values for one or more parameters (e.g., a parameter set) that define an aspect of the therapy delivered by IMD 372 according to that program. For example, a therapy stimulation program that controls delivery of stimulation by IMD 372 in the form of pulses may define values for voltage or current pulse amplitude, pulse width, pulse rate (e.g., pulse frequency), electrode combination, pulse shape, etc. for stimulation pulses delivered by IMD 372 according to that program. In some examples, parameters may include sequences of pulses, for example a “burst” of pulses with gradually increasing current magnitudes, or some other sequence. In some examples, IMD 372 may deliver therapy for a given duration and stop delivering therapy for a given duration. In other words, parameters of the electrical stimulation therapy may include an ON-time and an OFF-time. In some examples, an ON-time may be a few seconds or minutes and the OFF-time may also be for a few seconds or minutes. The ON-time may be equal to the OFF-time in some examples, while in other examples the ON-time and the OFF-time may be unequal durations.

Furthermore, IMD 372 may be configured to deliver control stimulation to patient 305 via a combination of electrodes of leads 330, alone or in combination with an electrode carried by or defined by an outer housing of IMD 372 to detect ECAP signals (e.g., control pulses and/or informed pulses). The tissue targeted by the stimulation may be the same or similar tissue targeted by the electrical stimulation therapy, but IMD 372 may deliver stimulation pulses for ECAP signal detection via the same, at least some of the same, or different electrodes. Since control stimulation pulses can be delivered in an interleaved manner with informed pulses (e.g., when the pulses configured to contribute to therapy interfere with the detection of ECAP signals or pulse sweeps intended for posture state detection via ECAP signals do not correspond to pulses intended for therapy purposes), a clinician and/or user may select any desired electrode combination for informed pulses. Like the electrical stimulation therapy, the control stimulation may be in the form of electrical stimulation pulses or continuous waveforms.

In one example, each control stimulation pulse may include a balanced, bi-phasic square pulse that employs an active recharge phase. However, in other examples, the control stimulation pulses may include a monophasic pulse followed by a passive recharge phase. In other examples, a control pulse may include an imbalanced bi-phasic portion and a passive recharge portion. Although not necessary, a bi-phasic control pulse may include an interphase interval between the positive and negative phase to promote propagation of the nerve impulse in response to the first phase of the bi-phasic pulse. The control stimulation may be delivered without interrupting the delivery of the electrical stimulation informed pulses, such as during the window between consecutive informed pulses. The control pulses may elicit an ECAP signal from the tissue, and IMD 372 may sense the ECAP signal via two or more electrodes on leads 330. In cases where the control stimulation pulses are applied to spinal cord 320, the signal may be sensed by IMD 372 from spinal cord 320.

In the example of FIG. 3, IMD 372 described as performing a plurality of processing and computing functions. However, external computing device 350, mobile computing device 354, wearable computing device 356 and/or servers 312 instead may perform one, several, or all of these functions. In this alternative example, IMD 372 functions to relay sensed signals to external computing device 350 for analysis, and external computing device 350 transmits instructions to IMD 372 to adjust the one or more parameters defining the electrical stimulation therapy based on analysis of the sensed signals.

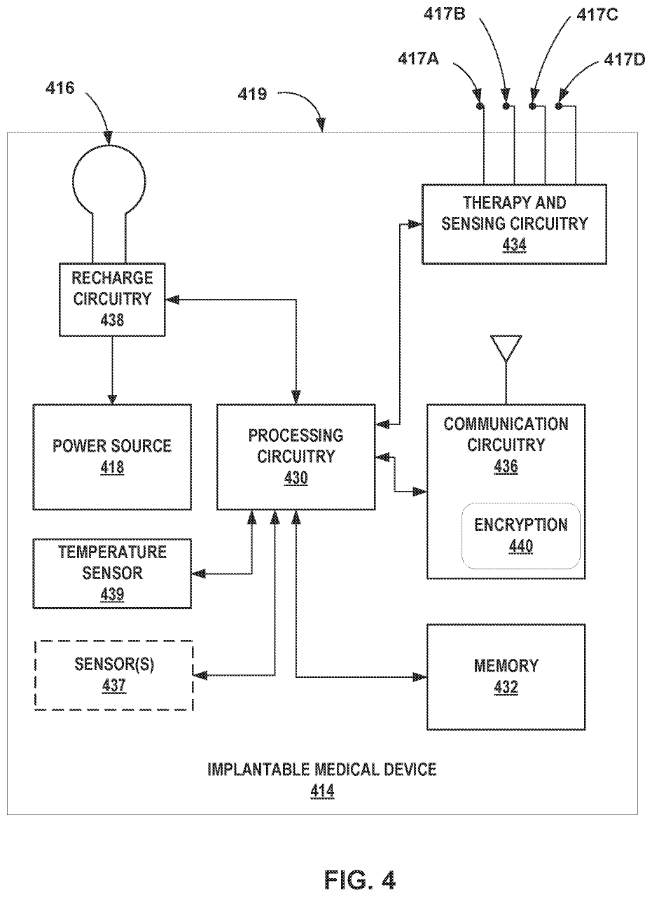

FIG. 4 is a block diagram illustrating example components of the medical device as described above. Implantable medical device 414 is an example of IMD 110 described above in relation to FIG. 1 and IMD 372 of FIG. 3 and may have the same or similar functions and characteristics. The medical device in the example of FIG. 4 is described as an implantable medical device, but the same functions, characteristics and techniques may also apply to other type of wearable or portable medical devices.

In the example illustrated in FIG. 4, IMD 414 includes temperature sensor 439, coil 416, processing circuitry 430, therapy and sensing circuitry 434, recharge circuitry 438, memory 432, communication circuitry 436, power source 418, and one or more sensors 437, such as an accelerometer. In other examples, IMD 414 may include a greater or a fewer number of components, e.g., in some examples, IMD 414 may not include temperature sensor 439 or sensors 437. In general, IMD 414 may comprise any suitable arrangement of hardware, alone or in combination with software and/or firmware, to perform the various techniques described herein attributed to IMD 414 and processing circuitry 430, and any equivalents thereof.

Processing circuitry 430 of IMD 414 may be implemented as one or more processors, such as one or more microprocessors, digital signal processors (DSPs), application specific integrated circuits (ASICs), field programmable gate arrays (FPGAs), or any other equivalent integrated or discrete logic circuitry, as well as any combinations of such components. IMD 414 may include a memory 432, such as random access memory (RAM), read only memory (ROM), programmable read only memory (PROM), erasable programmable read only memory (EPROM), electronically erasable programmable read only memory (EEPROM), flash memory, comprising executable instructions for causing the processing circuitry 430 to perform the actions attributed to this circuitry.

Moreover, although processing circuitry 430, therapy and sensing circuitry 434, recharge circuitry 438, communication circuitry 436, and temperature sensor 439 are described as separate modules, in some examples, some combination of processing circuitry 430, therapy and sensing circuitry 434, recharge circuitry 438, communication circuitry 436 and temperature sensor 439 are functionally integrated. In some examples, processing circuitry 430, therapy and sensing circuitry 434, recharge circuitry 438, communication circuitry 436, and temperature sensor 439 correspond to individual hardware units, such as ASICs, DSPs, FPGAs, or other hardware units. For example, components of IMD 414 may be implemented as separate circuits in some examples. In other examples, two or more components of IMD 414 may be implemented on a single integrated circuit, e.g., including processing circuitry 430, communication circuitry 436, memory 432, therapy and sensing circuitry 434, and so on. In this disclosure, therapy, and sensing circuitry 434 may be referred to as therapy circuitry 434, for simplicity.

Memory 432 may store therapy programs or other instructions that specify therapy parameter values for the therapy provided by therapy circuitry 434 and IMD 414. In some examples, memory 432 may also store temperature data from temperature sensor 439, instructions for recharging rechargeable power source 418, thresholds, instructions for communication between IMD 414 and an external computing device, or any other instructions required to perform tasks attributed to IMD 414. Memory 432 may be configured to store instructions for communication with and/or controlling one or more temperature sensors of temperature sensor 439. In various examples, memory 432 stores information related to determining the temperature of housing 19 and/or exterior surface(s) of housing 19 of IMD 414 based on temperatures sensed by one or more temperature sensors, such as temperature sensor 439, located within IMD 414.

For example, memory 432 may store programming settings such as parameters for electrical stimulation therapy output, e.g., magnitude, pulse width, and so on. Memory 432 may store parameters and other settings for the delivery of haptic stimulation. Settings may be individualized based on patient preference and/or patient physiology. For example, a stimulation intensity that is above the perception threshold for a first patient may be different than the stimulation intensity that may be above the perception threshold for a second patient. In some examples, a patient may find a particular frequency to be annoying or painful and therefore, may prefer a different frequency setting when receiving haptic stimulation as feedback.

Instructions stored at memory 432 when executed by processing circuitry 430 may determine whether a sensed bioelectrical signal is valid, such as and ECAP or other signal in response to an output electrical stimulation therapy event. Memory 432 may store programming instructions that when executed by processing circuitry 430 cause processing circuitry 430 to cause electrical stimulation circuitry therapy circuitry 434 to deliver electrical stimulation therapy to a target nerve of a patient. Memory 432 may also store instructions on encrypting and decrypting communications to be sent via communications circuitry 436 to an external computing device, as well as instructions for establishing a communication channel over a communication link, as described above in relation to FIGS. 1-3.

Therapy and sensing circuitry 434 may generate and deliver electrical stimulation under the control of processing circuitry 430. Therapy and sensing circuitry 434 may also output non-therapy stimulation, such as control pulses and haptic stimulation. In some examples, processing circuitry 430 controls therapy circuitry 434 by accessing memory 432 to selectively access and load at least one of the stimulation programs to therapy circuitry 434. For example, in operation, processing circuitry 430 may access memory 432 to load one of the stimulation programs to therapy circuitry 434. In such examples, relevant stimulation parameters may include a voltage amplitude, a current amplitude, a pulse rate, a pulse width, a duty cycle, or the combination of electrodes 417A, 417B, 417C, and 417D (collectively “electrodes 417”) that therapy circuitry 434 may use to deliver the electrical stimulation signal as well as sense biological signals. In other examples, IMD 414 may have more or fewer electrodes than the four shown in the example of FIG. 4. In some examples electrodes 417 may be part of or attached to a housing of IMD 414, e.g., a leadless electrode. In other examples, one or more of electrodes 417 may be part of a lead implanted in or attached to a patient to sense biological signals and/or deliver electrical stimulation, as described above in relation to FIG. 1.

In some examples, one or more electrodes connected to therapy circuitry 434 may connect to one or more sensing electrodes, e.g., attached to housing of IMD 414. In some examples the electrodes may be configured to detect an evoked motor response caused by the electrical stimulation therapy event, or other bioelectrical signals such as ECAPs, impedance and so on.

IMD 414 also includes components to receive power to recharge rechargeable power source 418 when rechargeable power source 418 has been at least partially depleted. As shown in FIG. 4, IMD 414 includes coil 416 and recharge circuitry 438 coupled to rechargeable power source 418. Recharge circuitry 438 may be configured to charge rechargeable power source 418 with the selected power level determined by either processing circuitry 430 or an external charging device, such as external computing IMD 110 described above in relation to FIG. 1. Recharge circuitry 438 may include any of a variety of charging and/or control circuitry configured to process or convert current induced in coil 416 into charging current to charge power source 418. For example, recharge circuitry 438 may include measurement circuitry configured to determine a magnitude of current received by secondary coil 416, a magnitude of current delivered to power source 418, and other measurements. Recharge circuitry 438 may send such measurements to processing circuitry 430 to be used in system metrics, and sent to an external computing device via communication circuitry 436.

Secondary coil 416 may include a coil of wire or other device capable of inductive coupling with a primary coil disposed external to the patient. Although secondary coil 416 is illustrated as a simple loop of in FIG. 4, secondary coil 416 may include multiple turns of conductive wire. Secondary coil 416 may include a winding of wire configured such that an electrical current can be induced within secondary coil 416 from a magnetic field. The induced electrical current may then be used to recharge rechargeable power source 418.

Recharge circuitry 438 may include one or more circuits that process, filter, convert and/or transform the electrical signal induced in the secondary coil to an electrical signal capable of recharging rechargeable power source 418. For example, in alternating current induction, recharge circuitry 438 may include a half-wave rectifier circuit and/or a full-wave rectifier circuit configured to convert alternating current from the induction to a direct current for rechargeable power source 418. The full-wave rectifier circuit may be more efficient at converting the induced energy for rechargeable power source 418. However, a half-wave rectifier circuit may be used to store energy in rechargeable power source 418 at a slower rate. In some examples, recharge circuitry 438 may include both a full-wave rectifier circuit and a half-wave rectifier circuit such that recharge circuitry 438 may switch between each circuit to control the charging rate of rechargeable power source 418 and temperature of IMD 414.

Rechargeable power source 418 may include one or more capacitors, batteries, and/or other energy storage devices. Rechargeable power source 418 may deliver operating power to the components of IMD 414. In some examples, rechargeable power source 418 may include a power generation circuit to produce the operating power. Rechargeable power source 418 may be configured to operate through many discharge and recharge cycles. Rechargeable power source 418 may also be configured to provide operational power to IMD 414 during the recharge process. In some examples, rechargeable power source 418 may be constructed with materials to reduce the amount of heat generated during charging. In other examples, IMD 414 may be constructed of materials and/or using structures that may help dissipate generated heat at rechargeable power source 418, recharge circuitry 438, and/or secondary coil 416 over a larger surface area of the housing of IMD 414.

Although rechargeable power source 418, recharge circuitry 438, and secondary coil 416 are shown as contained within the housing of IMD 414, in alternative implementations, at least one of these components may be disposed outside of the housing. For example, in some implementations, secondary coil 416 may be disposed outside of the housing of IMD 414 to facilitate better coupling between secondary coil 416 and the primary coil of external charging device. In other examples, power source 418 may be a primary power cell and IMD 414 may not include recharge circuitry 438 and recharge coil 416.

Processing circuitry 430 may also control the exchange of information with an external computing device using communication circuitry 436. Processing circuitry 430 may transmit operational information and receive therapy programs or therapy parameter adjustments over an established secure communication channel via communication circuitry 436. Also, in some examples, IMD 414 may communicate with other implanted devices, such as stimulators, control devices, or sensors, via communication circuitry 436. Communication circuitry 436 may include one or more antennas 437 configured to communicate with an external computing device, e.g., for power transfer or with the other devices. In addition, communication circuitry 436 may be configured to control the exchange of information related to sensed and/or determined temperature data, for example temperatures sensed by and/or determined from temperatures sensed using temperature sensor 439. In some examples, communication circuitry 436 may communicate using inductive communication, and in other examples, communication circuitry 436 may communicate using RF frequencies separate from the frequencies used for inductive charging.

In the example of FIG. 4, communication circuitry 436 includes circuitry 440 configured to manage encryption and decryption and may also execute some of the other functions related to establishing communications channels described in in this disclosure. In other examples, the encryption functions of circuitry 440 may be handled by processing circuitry 430, or by some other circuitry of IMD 414.

Communication circuitry 436 may be configured to support wireless communication. For example, communication circuitry 436 may be configured to support wireless communication using Bluetooth™ (e.g., BLE and other versions of Bluetooth™, including future versions of Bluetooth™), Wi-Fi™, Near-Field Communication (NFC), Near Field Magnetic Induction (NFMI), Long Term Evolution, 5th generation (LTE/5G), or MedRadio (MICS: Medical Implant Communication Service, MEDS: Medical External Device Service, MBAD: Medical Body Area Network)) between IMD 414 and another computing device, e.g., a computing device of system 100, 200 or 300 described above in relation to FIGS. 1-3. In some examples, communication circuitry 436 supports a communication frequency that may correspond to a high frequency or radio frequency, which may be a radio frequency established via Bluetooth, Wi-Fi, Near-Field Communication (NFC), 175 KHz inductive communication, or MICS, for example. Communication circuitry 436 may be configured to receive an inductive sting. Processing circuitry 430 of IMD 414 may receive, as updates to programs (e.g., at least one program parameter), values for various stimulation parameters such as magnitude and electrode combination, from an external computing device via communication circuitry 436. In addition, communication circuitry 436 may communicate with an external medical device via proximal inductive interaction of IMD 414, e.g., during recharging. Communication circuitry 436 may send and receive information on a continuous basis, at periodic intervals, or upon request from an external computing device. In some examples, communication circuitry 436 may also be referred to as telemetry circuitry in this disclosure.

In some examples, processing circuitry 430 may transmit additional information to external charging device related to the operation of rechargeable power source 418, e.g., at a more limited authentication level than for sending and receiving operational programming instructions or parameters. For example, processing circuitry 430 may use communication circuitry 436 to transmit indications that rechargeable power source 418 is completely charged, rechargeable power source 418 is fully discharged, the amount of charging current output by recharge circuitry 438 e.g., to power source 418, or any other charge status of rechargeable power source 418. In some examples, processing circuitry 430 may use communication circuitry 436 to transmit instructions to the external charging device, including instructions regarding further control of the charging session, for example instructions to lower the power level or to terminate the charging session, based on the determined temperature of the housing/external surface 419 of the IMD.

Processing circuitry 430 may also transmit information to external charging device that indicates any errors with rechargeable power source 418 that may prevent rechargeable power source 418 from providing operational power to the components of IMD 414. In various examples, processing circuitry 430 may receive, through communication circuitry 436, instructions for algorithms, including formulas and/or values for constants to be used in the formulas that may be used to determine the temperature of the housing 419 and/or exterior surface(s) of housing 419 of IMD 414 based on temperatures sensed by temperature sensor 439 located within IMD 414 during and after a recharging session performed on rechargeable power source 418.

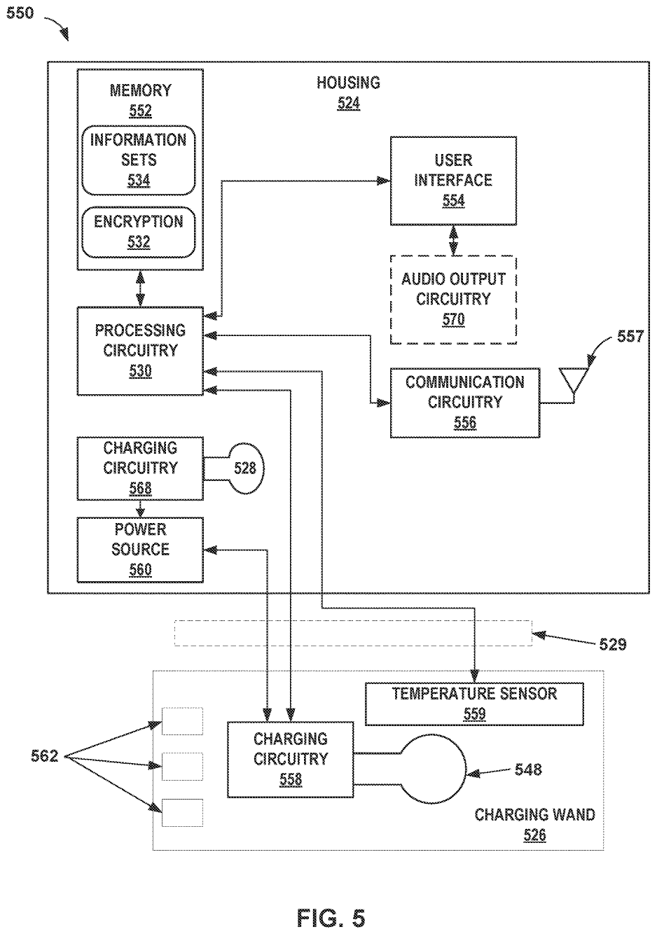

FIG. 5 is a block diagram of an example an external computing device of FIGS. 1 and 3. External charging device 550 in of FIG. 5 is an example of external computing device 150, 250, and 350 described above in relation to FIGS. 1-3 and may have the same or similar functions. As described above, in some examples, external charging device 550 may be a hand-held device, while in other examples, external charging device 550 may be a larger or a non-portable device. In addition, in other examples external charging device 550 may be included as part of an external programmer or include functionality of an external programmer. As shown in the example of FIG. 5, external charging device 550 includes two separate components. Housing 524 encloses components such as a processing circuitry 530, memory 552, user interface 554, communication circuitry 556, power button, audio output circuitry 570 and power source 560. Charging head 526, also referred to as a charging wand 526, may include charging circuitry 558, temperature sensor 559, and coil 548. Housing 524 is electrically coupled to charging head 526 via charging cable 529. In some examples, housing 524 may also include charging circuitry 568 and coil 528, which is an example of coil 128 described above in relation to FIG. 1.

In some examples, separate charging wand 526 may facilitate positioning of coil 548 over coil 116 of IMD 110 of FIG. 1, or coil 416 of FIG. 4. In some examples, charging circuitry 568 and/or coil 528 may be integrated within housing 524. In other examples, external charging device 550 may not include charging wand 526. Coil 548 and coil 528 may also be referred to as an antenna.

External charging device 550 may also include one or more temperature sensors, illustrated as temperature sensor 559, similar to temperature sensor 439 of FIG. 4. As shown in FIG. 5, temperature sensor 559 may be disposed within charging head 526. For example, charging head 526 may include one or more temperature sensors positioned and configured to sense the temperature of coil 548 and/or a surface of the housing of charging head 526. In some examples, external charging device 550 may not include temperature sensor 559. In other examples, one or more temperature sensors of temperature sensor 559 may be disposed within housing 524, such as located to sense the temperature of primary coil 528 and/or charging circuitry 568.

In general, external charging device 550 comprises any suitable arrangement of hardware, alone or in combination with software and/or firmware, to perform the techniques ascribed to external charging device 550, and processing circuitry 530, user interface 554, communication circuitry 556, and charging circuitry 558 of external charging device 550, and/or any equivalents thereof. In various examples, external charging device 550 may include one or more processors, such as one or more microprocessors, DSPs, ASICs, FPGAs, or any other equivalent integrated or discrete logic circuitry, as well as any combinations of such components.

Similar to IMD 110 and 414 described above in relation to FIGS. 1 and 4, components of external charging device 550 shown in FIG. 5 may be implemented as separate circuitry, or combined into one or more integrated circuits. In other words, although processing circuitry 530, communication circuitry 556, charging circuitry 558, and temperature sensor 559 are described as separate modules, in some examples, processing circuitry 530, communication circuitry 556, charging circuitry 558, and/or temperature sensor 559 are functionally integrated. In some examples, processing circuitry 530, communication circuitry 556, charging circuitry 558, and/or temperature sensor 559 correspond to individual hardware units, such as ASICs, DSPs, FPGAs, or other hardware units.

External charging device 550 also, in various examples, may include a memory 552, such as RAM, ROM, PROM, EPROM, EEPROM, flash memory, a hard disk, a CD-ROM, comprising executable instructions for causing the one or more processors to perform the actions attributed to external charging device 550. Memory 552 may store instructions that, when executed by processing circuitry 530, cause processing circuitry 530 and external charging device 550 to provide the functionality ascribed to external charging device 550 throughout this disclosure, and/or any equivalents thereof, including information sets 534 for the different authentication levels, as described above in relation to FIG. 1. For example, memory 552 may include instructions that cause processing circuitry 530 to control the power level used to charge IMD 414 of FIG. 4, as communicated from IMD 414 via a communication channel, e.g., communication channel 0 (204) described above in relation to FIG. 2. Memory 552 may include a record of selected power levels, sensed temperatures, determined temperatures, or any other data related to charging rechargeable power source 418, described above in relation to FIG. 4.

User interface 554 may include buttons, a keypad, indicator lights, a microphone for voice commands, a display, such as a liquid crystal (LCD), light-emitting diode (LED), or cathode ray tube (CRT) and audio output circuitry.

In some examples, the display of user interface 554 may be a touch screen. As discussed in this disclosure, processing circuitry 530 may present and receive information relating to the charging of rechargeable power source 418 via user interface 554. For example, user interface 554 may indicate when charging is occurring, quality of the alignment between primary coil 528 or 548 and the secondary coil of the IMD, the selected power level, current charge level of power source 418, duration of the current recharge session, anticipated remaining time of the charging session, sensed temperatures, or any other information. In some examples, processing circuitry 530 may receive some of the information displayed on user interface 554, e.g., via communication circuitry such as communication circuitry 556. In some examples, user interface 554 may provide an indication to the user regarding the quality of alignment between coil 416, depicted in FIG. 4 and coil 548, based on one or more system metrics, such as the charge current to the battery of the IMD.

Processing circuitry 530 may also receive user input via user interface 554. The input may be, for example, in the form of pressing a button on a keypad or selecting an icon from a touch screen. The input may change programmed settings, start, or stop therapy, request starting or stopping a recharge session, a desired level of charging, or one or more statistics related to charging power source 418 (e.g., the cumulative thermal dose). In this manner, user interface 554 may allow the user to view information related to the operation of IMD 414.

Charging circuitry 558 may include one or more circuits that generate an electrical current within primary coil 548. Charging circuitry 558 may generate an alternating current of specified amplitude and frequency in some examples. In other examples, charging circuitry 558 may generate a direct current. In any case, charging circuitry 558 may be capable of generating electrical signals, and subsequent magnetic fields, to transmit various levels of power to IMD 414. In this manner, charging circuitry 558 may be configured to charge power source 418 of IMD 414 with the selected power level.

Power source 560 may deliver operating power to the components of external charging device 550. Power source 560 may also deliver the operating power to drive primary coil 548 or primary coil 528 during the charging process. Power source 560 may include a battery and a power generation circuit to produce the operating power. In some examples, a battery of power source 560 may be rechargeable to allow extended portable operation. In other examples, power source 560 may draw power from a wired voltage source such as a consumer or commercial power outlet.

Processing circuitry 530 may, when requested, transmit any stored data in memory 552 to another computing device for review or further processing, such as to servers 112 depicted in FIG. 1. Processing circuitry 530 may be configured to access memory, such as memory 432 of IMD 414 and/or memory 552 of external charging device 550, to retrieve information comprising instructions, formulas, and determined values for one or more constants. As described above in relation to FIGS. 1-4, processing circuitry 530 and communication circuitry 556 may establish one or more secure communication channels over a communication link. In some examples, processing circuitry 530 may encrypt and decrypt transferred information during a communication session, based encryption keys associated with the authentication level for a respective communication channel. In the example of FIG. 5, instructions for establishing the communication channel, handling the encryption handshaking, setting, and resetting the communication session timers, storing encryption keys and other similar functions may be stored at memory 552 (e.g., encryption 532). In other examples, (not shown in FIGS. 4 and 5) the encryption keys and/or encryption instructions may be stored in a separate encrypted memory in communication with processing circuitry 430 or 530.

In some examples, communication circuitry 556 may establish a first communication channel directly to an implantable medical device, e.g., IMD 210 of FIG. 2. Communication circuitry 556 may establish a second communication channel directly to the implantable medical device, based on a different authentication level than for the first communication channel. In some examples another computing device, e.g., of systems 100, 200 or 300 described above in relation to FIGS. 1-3, may transfer information with IMD 210 via communication channel through communication 1 (206) via external recharging device 250.

Communication circuitry 556 may support wireless communication between IMD 110 of FIG. 1 and external charging device 550 under the control of processing circuitry 530. Communication circuitry 556 may also be configured to communicate with another computing device via wireless communication techniques, or direct communication through a wired connection. In some examples, communication circuitry 556 may be substantially similar to communication circuitry 436 of IMD 414 described herein, providing wireless communication via an RF or proximal inductive medium. In some examples, communication circuitry 556 may include an antenna 557, which may take on a variety of forms, such as an internal or external antenna. Although communication circuitry 556 may each include dedicated antennas for communications between these devices, communication circuitry 556 and 436 may instead, or additionally, be configured to utilize inductive coupling from coils 416 and 548 to transfer data.

Examples of local wireless communication techniques that may be employed to facilitate communication between external charging device 550 and IMD 414 include radio frequency and/or inductive communication according to any of a variety of standard or proprietary communication protocols, or according to other communication protocols such as the IEEE 802.11x or Bluetooth specification sets. In this manner, other external devices may be capable of communicating with external charging device 550 without needing to establish a secure wireless connection.

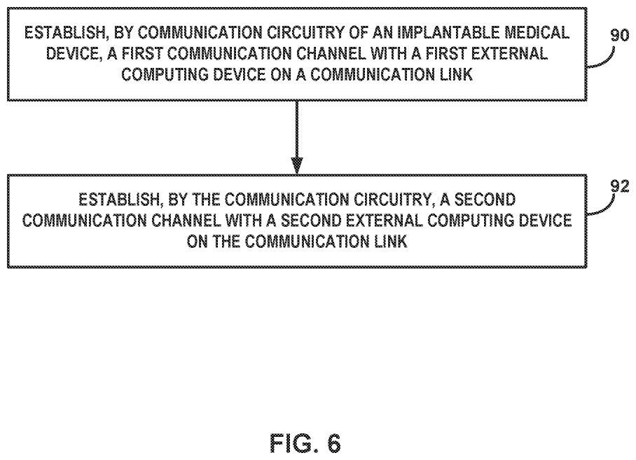

FIG. 6 is a flow chart illustrating an example mode of operation of the communication circuitry of the systems of this disclosure. The flow chart in the example of FIG. 6 may apply to any of systems 100, 200 and 300 described above in relation to FIGS. 1-3 and to implantable medical device 414 and to external computing device 550 described above in relation to FIGS. 4 and 5. As discussed above, the techniques of this disclosure may also apply to a medical device, such as a wearable medical device, in addition to an implantable medical device.

In some examples, and as shown in FIG. 5, processing circuitry 530 may cause communication circuitry 556 to establish a first communication channel with communication circuitry 436 of implantable medical device 414 on a communication link (90), such as communication link 1 (202) shown in FIG. 2 between IMD 210 and external computing device 250. In some examples, communication circuitry 556 may establish a communication channel, e.g., communication channel 0 (204) directly to communication circuitry 436. Communication circuitry 556 may transfer information between communication circuitry 436 based on an information set associated with the authentication level of communication channel 0 (204), e.g., power transfer information, in the example of a recharger, or limited parameter adjustments in the example of a patient programmer.

In some examples, communication circuitry 556 may also establish a second communication channel, e.g., communication channel 1 (206) directly with communication circuitry 436 on the same communication link (92). In some examples, communication circuitry 556 and communication circuitry 436 may further establish three or more communication channels, which in some examples may each have a separate associated authentication level and associated encryption handshaking. Communication circuitry 556 and communication circuitry 436 may share the bandwidth of communication link 1 (202) over the separate communication channels by using time division.

During the communication session, the processing circuitry of any of systems 100, 200 and 300, e.g., processing circuitry of external communication device 150 and processing circuitry of IMD 110 depicted in FIG. 1, may select which information to send and process based on the information set associated with the authentication level for the established channel. The processing circuitry may cause the sent information to be encrypted with the encryption key associated with the communication channel.

In some examples, other computing devices, e.g., programmer 214 of system 200, servers 312 of system 300, or other computing devices, may transfer information to communication circuitry 436 and IMD 414 via communication circuitry 556 and communication link 1 (202). In other examples, as shown in FIGS. 1 and 3, the other computing devices of systems 100 and 300 may establish communication channels directly with the medical device.

The different authentication levels described herein may be associated with certain actions or features that the device (e.g., an IMD) is authorized to perform. In other words, an IMD may perform an action when the request for that action is received via communication having the authentication level that includes that action. For example, a first authentication level may be assigned to a clinician programmer such that the first authentication level enables any actions related to adjusting therapy or therapy schedules of the IMD, reading or writing to different program groups, receiving IMD battery status, or other actions related to programming the IMD. As another example, a second authentication level may be assigned to an external recharger such that the second authentication level enables checking the IMD compatibility or writing to recharge logs, but does not enable changing any therapy programs or therapy schedules.

The IMD, other medical device, or even external devices that can communicate within the overall system (e.g., an external programmer, recharger, or networked device), may store a list, chart, or table that identifies actions or features, or even one or more sub-commands for each action or feature, associated with different authorization levels. In this manner, the IMD or other devices of the system can only perform actions, features, or commands in response to a request received via the authentical level associated with that specific action, feature, or command. The different authentication levels may thus enable, or restrict, various actions from being performed depending on which devices (or user profiles of a device) submitted the request.

Each system of devices (or a single device) may be able to perform N number of actions. Example actions for a medical device system, such as system 100 of FIG. 1, may include actions such as IMD identification, IMD compatibility check, read from instrument memory, write to instrument memory, read battery status and recharge information, set state of charge, write recharge logs, read time, write time, read and change therapy parameters, read and change a scheduled therapy, or even perform a firmware update. Each of these actions may be associated with one, two, three, or more different commands (e.g., M number of commands) that may request the device to perform some aspect, or combination of aspects, associated with the action. In some examples, the authorization level may have access to all commands for that action, be restricted from any commands for that action, or have access to one or more, but not all, of the commands for the action. For various patient or system risks, different authentication levels may be restricted from one or more of these actions and/or commands. Some commands, for example, may impose a different magnitude or severity of risk when compared to other commands (e.g., a command to turn on stimulation or increase amplitude may have more risk than turning off stimulation or decreasing amplitude). In some examples, authentication levels may have conditional access or restriction to one or more actions or commands, and the memory may retain these conditions as appropriate.

In one example, IMD 414 may include memory 432 that is configured to store a first list of authorized actions for the first authentication level and a second list of authorized actions for the second authentication level, where the first list of authorized actions is different from the second list of authorized actions. In this manner, some actions may be associated with the first authentication level, and other actions may be associated with the second authentication level. The lists may have completely different actions, or the lists may have some common actions. Processing circuitry 430 may then be configured to authorize performance of an action for IMD 414 as requested by one of the first information or the second information according to the respective first list of authorized actions for the first authentication level or second list of authorized actions for the second authentication level.

An authentication level table, for example, may store these relationships between authentication levels and authorized actions (or commands of each action). In some examples, the different authentication levels may be listed. In other examples, different authentication levels may be represented by the respective device that uses that authentication level, such as clinician programmer, patient programmer, remote server, recharger device, etc. In this manner, each device may be directly associated with available and unavailable actions and/or commands. In some examples, this table may establish rules for the sending device, but in other examples, the receiving device may use the table to allow or not allow various received requests.

An example table is shown below in Table 1. For example, programmers may have an authorization level 1, a recharger may have an authorization level 2, and a firmware update channel may be associated with authorization level 3. A “YES” label indicates that the level is authorized to request the listed action or command. A “NO” label indicates that the level is not authorized to request the listed action or command.

TABLE 1
Example actions and commands for different authorization levels. More
or fewer actions, commands, or levels may be used in other example
tables as appropriate for the system function (e.g., type of therapy)
and types of devices that may communicate with each other.

| | | Authoriza- | Authoriza- | Authoriza- |
|---|---|---|---|---|
| | | tion | tion | tion |
| Action | Command | level 1 | level 2 | level 3 |
| IMD | A command | YES | YES | YES |
| compatibility | B command | | | |
| check | | | | |
| Read battery | C command | YES | NO | NO |
| status | D command | | | |
| Write recharge | E command | NO | NO | NO |
| log | | | | |
| Read/write | F, G, H, I | YES | NO | NO |
| therapy | command | | | |
| program | | | | |
| Read/write | J, K, L, M | YES | NO | NO |
| therapy | command | | | |
| schedule | | | | |
| Perform | N, O, P, Q | NO | NO | YES |
| firmware | command | | | |
| update | | | | |

The techniques of this disclosure may also be described in the following examples.

Example 1: An implantable medical device comprising communication circuitry configured to: establish a first communication channel with a first external computing device on a communication link; establish a second communication channel with a second external computing device on the communication link, wherein the first communication channel transfers first information at a first authentication level on the communication link, and wherein the second communication channel transfers second information at a second authentication level different than the first authentication level on the same communication link.

Example 2: The device of example 1, wherein the communication circuitry is further configured to establish the first communication channel and the second communication channel on the communication link using time division multiplexing.

Example 3: The device of any of examples 1 and 2, wherein the first communication channel is a first secure communication channel established based on a first encryption key, and wherein the second communication channel is a second secure communication channel established based on a second encryption key.

Example 4: The device of example 3, wherein the communication circuitry is configured to establish the first secure communication based on the first encryption key for the duration of a communication session.

Example 5: The device of any of examples 1 through 4, wherein the first external computing device establishes the first communication channel directly to the implantable medical device, wherein the first external computing device further establishes the second communication channel directly to the implantable medical device, and wherein the second external computing device transfers information on the second communication channel via the first external computing device.

Example 6: The device of example 5, wherein the communication link is a first communication link, wherein the second external computing device communicates with the first external computing device via a second communication link different than the first communication link.

Example 7: The device of example 6, wherein the first communication link operates with a first protocol, and wherein the second communication link operates with a second protocol different from the first protocol.

Example 8: The device of any of examples 1 through 7, wherein the first authentication level is configured to communicate with a first information set; wherein the second authentication level is configured to communicate with an expanded information set comprising more elements than the first information set.

Example 9: The device of example 8, wherein the first information set and the expanded information set comprise one or more of: sensed data, operating status, operating commands, one or more parameters defining the operation of sensing circuitry configured to detect biological signals from a patient, or one or more parameters defining therapy deliverable by the implantable medical device.

Example 10. The device of any of examples 1 through 9, further comprising: a memory configured to store a first list of authorized actions for the first authentication level and a second list of authorized actions for the second authentication level, wherein the first list of authorized actions is different from the second list of authorized actions; and processing circuitry configured to authorize performance of an action for the device as requested by one of the first information or the second information according to the respective first list of authorized actions for the first authentication level or second list of authorized actions for the second authentication level.

Example 11. The device of any of examples 1 through 10, wherein the device is an implantable medical device.

Example 12. The device of example 11, further comprising therapy circuitry configured to deliver electrical stimulation therapy to a patient.

Example 13. The device of example 11, further comprising sensing circuitry configured to sense a signal from a patient.

Example 14: A method comprising establishing, by communication circuitry of an implantable medical device, a first communication channel with a first external computing device on a communication link; establishing, by the communication circuitry, a second communication channel with a second external computing device on the communication link, wherein the first communication channel transfers first information at a first authentication level, and wherein the second external computing device transfers second information on the second communication channel via the first external computing device at a second authentication level different than the first authentication level.

Example 15: The method of example 14, further comprising sharing, by the communication circuitry bandwidth for the first communication channel and the second communication channel of the communication link using time division multiplexing.

Example 16: The method of any of examples 14 and 15, establishing the first communication channel as a first secure communication channel established based on a first encryption key, and establishing the second communication channel as a second secure communication channel based on a second encryption key.

Example 17: The method of example 16, wherein establishing the first secure communication channel comprises establishing the first secure communication channel for the duration of a communication session.

Example 18: The method of any of examples 14 through 17, wherein the first external computing device establishes the first communication channel directly to the implantable medical device, wherein the first external computing device further establishes the second communication channel directly to the implantable medical device, and wherein the second external computing device transfers information on the second communication channel via the first external computing device.

Example 19: The method of example 18, wherein the communication link is a first communication link, wherein the second external computing device communicates with the first external computing device via a second communication link different than the first communication link.

Example 20: The method of example 19, wherein the first communication link operates with a first protocol, and wherein the second communication link operates with a second protocol different from the first protocol.

Example 21: The method of any of examples 14 through 20, wherein the first authentication level is configured to communicate with a first information set; wherein the second authentication level is configured to communicate with an expanded information set comprising more elements than the first information set.

Example 22: The device of example 21, wherein the first information set and the expanded information set comprise one or more of: sensed data, operating status, operating commands, one or more parameters defining the operation of sensing circuitry configured to detect biological signals from a patient, or one or more parameters defining therapy deliverable by the implantable medical device.

Example 23: A non-transitory computer-readable storage medium comprising instructions that, when executed, cause one or more processors of a computing device to: cause communication circuitry of an implantable medical device to establish a first communication channel with a first external computing device on a communication link; cause communication circuitry of the implantable medical device to establish a second communication channel with a second external computing device on the communication link, wherein the first communication channel transfers first information at a first authentication level, and wherein the second external computing device transfers second information on the second communication channel via the first external computing device at a second authentication level different than the first authentication level.

Example 24: The non-transitory computer-readable storage medium of example 19, further comprising instructions for causing a programmable processor to: cause the communication circuitry to establish the first communication channel and the second communication channel on the communication link using time division multiplexing, wherein the first communication channel is a first secure communication channel established based on a first encryption key, and wherein the second communication channel is a second secure communication channel established based on a second encryption key.

Example 25: An implantable medical device comprising communication circuitry configured to: establish a first communication channel with a first external computing device on a first communication link; establish a second communication channel with a second external computing device on a second communication link, wherein the first communication channel transfers first information at a first authentication level on the first communication link, and wherein the second communication channel transfers second information at a second authentication level different than the first authentication level on the second communication link.

Example 26: The implantable medical device of example 25, wherein the communication circuitry is further configured to establish a third communication channel on the first communication link, wherein the third communication channel transfers third information at a third authentication level, wherein the first communication channel and the third communication channel operate during the same communication session and share the bandwidth of the first communication link over the separate communication channels by using time division.

Example 27: The implantable medical device of example 25, wherein the communication circuitry is further configured to establish a fourth communication channel on the second communication link, wherein the fourth communication channel transfers fourth information at a fourth authentication level, wherein the second communication channel and the fourth communication channel operate during the same communication session and share the bandwidth of the second communication link over the separate communication channels by using time division.

In one or more examples, the functions described above may be implemented in hardware, software, firmware, or any combination thereof. For example, the various components of FIGS. 1-4, processing circuitry 430 and processing circuitry 530 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor” and “processing circuitry,” as used herein, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples of the disclosure have been described. These and other examples are within the scope of the following claims.

1. A medical device, the device comprising:
communication circuitry configured to:
establish a first communication channel with a first external computing device on a communication link;
establish a second communication channel with a second external computing device on the communication link,
wherein the first communication channel transfers first information at a first authentication level on the communication link, and
wherein the second communication channel transfers second information at a second authentication level different than the first authentication level on the same communication link.

2. The device of claim 1, wherein the communication circuitry is further configured to establish the first communication channel and the second communication channel on the communication link using time division multiplexing.

3. The device of claim 1,
wherein the first communication channel is a first secure communication channel established based on a first encryption key, and
wherein the second communication channel is a second secure communication channel established based on a second encryption key.

4. The device of claim 3, wherein the communication circuitry is configured to establish the first secure communication based on the first encryption key for the duration of a communication session.

5. The device of claim 1,
wherein the first external computing device establishes the first communication channel directly to the implantable medical device,
wherein the first external computing device further establishes the second communication channel directly to the implantable medical device, and
wherein the second external computing device transfers information on the second communication channel via the first external computing device.

6. The device of claim 5,
wherein the communication link is a first communication link,
wherein the second external computing device communicates with the first external computing device via a second communication link different than the first communication link.

7. The device of claim 6,
wherein the first communication link operates with a first protocol, and
wherein the second communication link operates with a second protocol different from the first protocol.

8. The device of claim 1,
wherein the first authentication level is configured to communicate with a first information set;
wherein the second authentication level is configured to communicate with an expanded information set comprising different elements than the first information set.

9. The device of claim 8, wherein the first information set and the expanded information set comprise one or more of: sensed data, operating status, operating commands, one or more parameters defining the operation of sensing circuitry configured to detect biological signals from a patient, identifying information, power information, firmware update commands, memory access commands, or one or more parameters defining therapy deliverable by the implantable medical device.

10. A method comprising:
establishing, by communication circuitry of a medical device, a first communication channel with a first external computing device on a communication link;
establishing, by the communication circuitry, a second communication channel with a second external computing device on the communication link,
wherein the first communication channel transfers first information at a first authentication level, and
wherein the second external computing device transfers second information on the second communication channel via the first external computing device at a second authentication level different than the first authentication level.

11. The method of claim 10, further comprising sharing, by the communication circuitry bandwidth for the first communication channel and the second communication channel of the communication link using time division multiplexing.

12. The method of claim 10,
establishing the first communication channel as a first secure communication channel established based on a first encryption key, and
establishing the second communication channel as a second secure communication channel based on a second encryption key.

13. The method of claim 12, wherein establishing the first secure communication channel comprises establishing the first secure communication channel for the duration of a communication session.

14. The method of claim 10,
wherein the first external computing device establishes the first communication channel directly to the implantable medical device,
wherein the first external computing device further establishes the second communication channel directly to the implantable medical device, and
wherein the second external computing device transfers information on the second communication channel via the first external computing device.

15. The method of claim 14,
wherein the communication link is a first communication link,
wherein the second external computing device communicates with the first external computing device via a second communication link different than the first communication link.

16. The method of claim 15,
wherein the first communication link operates with a first protocol, and
wherein the second communication link operates with a second protocol different from the first protocol.

17. The method of claim 10,
wherein the first authentication level is configured to communicate with a first information set;
wherein the second authentication level is configured to communicate with an expanded information set comprising more elements than the first information set.

18. The device of claim 17, wherein the first information set and the expanded information set comprise one or more of: sensed data, operating status, operating commands, one or more parameters defining the operation of sensing circuitry configured to detect biological signals from a patient, or one or more parameters defining therapy deliverable by the implantable medical device.

19. A non-transitory computer-readable storage medium comprising instructions that, when executed, cause one or more processors of a computing device to:
cause communication circuitry of a medical device to establish a first communication channel with a first external computing device on a communication link;
cause communication circuitry of the implantable medical device to establish a second communication channel with a second external computing device on the communication link,
wherein the first communication channel transfers first information at a first authentication level, and
wherein the second external computing device transfers second information on the second communication channel via the first external computing device at a second authentication level different than the first authentication level.

20. The non-transitory computer-readable storage medium of claim 19, further comprising instructions for causing a programmable processor to:
cause the communication circuitry to establish the first communication channel and the second communication channel on the communication link using time division multiplexing,
wherein the first communication channel is a first secure communication channel established based on a first encryption key, and
wherein the second communication channel is a second secure communication channel established based on a second encryption key.

21. The non-transitory computer-readable storage medium of claim 19, wherein the medical device is a neurostimulation device and the second external computing device is a patient programmer.
